(19) United States
(12) Patent Application Publication
(10) Pub. No.: US 2009/0093773 A1
(43) Pub. Date: Apr. 9, 2009

(54) Unit-matching assisting tool for quantitative injection of medicine and method for adjusting injection rate of medicine

(75) Inventor: Kenichi Toraishi, Fukuoka-shi (JP)

Correspondence Address: JORDAN AND HAMBURG LLP, 122 EAST 42ND STREET, SUITE 4000, NEW YORK, NY, 10168, US

(73) Assignees: Kenichi Toraishi, Fukuoka-shi (JP); Yuko Toraishi, Fukuoka-shi (JP)

(21) Appl. No.: 12/286,180

(22) Filed: Sep. 29, 2008

(30) Foreign Application Priority Data
Oct. 5, 2007 (JP) 2007-262373

(51) Int. Cl. A61M 5/00

(52) U.S. Cl. 604/246

(57) ABSTRACT

The axial member is moved in the direction opposite to the main body (backward direction) by turning right the gripping member of the insulin injector. Next, the axial member is fitted in the recessed portions formed in the support structures of the unit-matching assisting tool for quantitative injection of medicine, and the gripping member is turned left in the turning direction, wherein the axial member is moved to the (forward direction) to the main body. And, the gripping member is turned left in the turning direction until the support structures are brought into contact with the main body and the gripping member, and the axial member is thereby moved forward. In this state, the indication of the dial is an injection rate of medicine.

### BACKGROUND OF THE INVENTION

(1) Field of the Invention

The present invention relates to a unit-matching assisting tool for quantitative injection of medicine and a method for adjusting an injection rate of medicine. In detail, the invention pertains to a unit-matching assisting tool for quantitative injection of medicine, in which a medicine quantitative injector is fitted, for assisting in the quantitative injection of medicine, and a method for adjusting an injection rate of medicine by which the injection rate of medicine by a medicine quantitative injector is adjusted.

(2) Description of Related Art

There may be a case where a diabetic patient is in a state where he or she can use only a single hand due to cerebrovascular disorder, gangrene, bone fracture resulting from a fall, or paralysis on one side of his or her body, etc. For such a patient who can use only a single hand, an assisting tool to carry out hypodermic injection of insulin oneself is required.

Several types of such an assisting tool have been proposed. For example, JP2007-14615(A) describes an insulin injection assisting tool as shown in FIG. 4. That is, an insulin injection assisting tool 101 described in JP2007-14615(A) includes a needle case fixing device 102 for fixing a needle case of a pen-type insulin injector 106 and a needle cap fixing device 103 for fixing a needle cap of the insulin injector 106. Also, JP2007-14615(A) describes, as a method for using such an insulin injection assisting tool, a method by which the insulin injector assisting tool 101 is fixed on a desk, next, a needle case 104 is placed in the insulin injection assisting tool 101, the insulin injector 106 is connected to an injection needle in the needle case 104, the insulin injector 106 to which an injection needle with a needle cap is connected is separated from the needle case 104 fixed at the insulin injection assisting tool 101, and the needle cap is removed with the needle cap of the insulin injector 106 fixed at the needle cap fixing device 103.

### BRIEF SUMMARY OF THE INVENTION

However, a diabetic patient carries out hypodermic injection of a predetermined rate of insulin. The insulin rate is appropriately increased and decreased according to the symptom and laboratory findings.

Therefore, as a pen-type insulin injector by which an insulin rate according to respective patients and symptoms can be injected, a pen-type insulin injector as shown in FIG. 2 is available.

That is, as shown in FIG. 2, a pen-type insulin injector 21 is provided with a main body 22 having a Dial indication opening portion 23 formed thereon, an axial member 24 having a substantially circular section, which is inserted from one end of the main body 22 into the main body 22, has a dial 25 displayed thereon, and is movable in the forward and backward direction by turning, a gripping member 26 attached to one end of the axial member, in which the length in the perpendicular direction with respect to the lengthwise direction of the axial member is longer than the outer diameter of the axial member, an injection button 27 for injecting insulin, which is attached to the gripping member, an accommodating body 28 attached to the other end of the main body, in which an insulin cartridge 28A having insulin filled therein is accommodated, and a injection needle attaching portion 29 to which an injection needle 30 is attached.

Also, an insulin injector based on a type in which the quantity of injected insulin is increased and decreased according to an increase or a decrease in the graduation of such a dial is appropriate for normal diabetic patients. However, it becomes inconvenient in the following cases. That is, 10% to 20% of diabetic patients of the total number of diabetic patients suffer from a lowering in eyesight, wherein it is difficult to set the dial to a desired graduation by turning the dial, and if the graduation cannot be observed, it is not possible for a diabetic patient to inject oneself. Therefore, it is necessary that someone assists in carrying out the insulin injection for the diabetic patient. Accordingly, if the diabetic patient attempts to have someone inject insulin, persons authorized to inject insulin by law other than the diabetic patient are only a medical doctor and a nurse. If the diabetic patient calls for a medical doctor or a nurse to his or her house and has the doctor or nurse carrying out an injection, the need to pay for medical expenses arises, wherein such a problem occurs by which an increase in medical expenses is brought about in line with an increase in the number of injections.

The present invention was developed in view of the above-described shortcomings and problems, and it is therefore an object of the invention to provide a unit-matching assisting tool for quantitative injection of medicine by which even a diabetic patient suffering from a lowering in eyesight can self-inject a necessary fixed volume of medicine, and a method for adjusting an injection rate of medicine using the unit-matching assisting tool for quantitative injection of medicine.

In order to achieve the above-described object, the unit-matching assisting tool for quantitative injection of medicine according to the present invention is featured in that the unit-matching assisting tool has a medicine quantitative injector fitted therein and assists quantitative injection of medicine, wherein the medicine quantitative injector includes: a main body; an axial member, the section of which is substantially circular, inserted into the corresponding main body and movable in the forward and backward direction by turning; and a gripping member attached to one end of the axial member and having a longer length in the perpendicular direction with respect to the lengthwise direction of the axial member than the outer diameter of the axial member, and the unit-matching assisting tool further includes a supporting member composed of a plurality of support structures into which the axial member is fitted and which have recessed portions having a smaller gap than the lengths of the gripping member and the main body in the perpendicular direction with respect to the lengthwise direction of the axial member, and wherein the support structures are detachably coupled with each other, in which the recessed portions are made continuous.

Herein, a support member can be brought into contact with the gripping member and the main body by the support member composed of a plurality of support structures in which recessed portions having the axial member fitted therein and having a smaller gap than the length in the perpendicular direction with respect to the lengthwise direction of the axial member of the gripping members and the main body are formed, respectively.

Also, it is possible to increase or decrease the number of the supporting structures by making the recessed portions continuous and detachably coupling them to each other.

Also, in the unit-matching assisting tool for quantitative injection of medicine according to the present invention, where at least one of the support structures is provided with the first area having the recessed portion formed therein and the second area substantially perpendicular with respect to the first area, the supporting member is placed on a desk or a table, the axial member of the medicine quantitative injector is fitted in the recessed portions.

In addition, in the unit-matching assisting tool for quantitative injection of medicine according to the present invention, where the support structures have substantially the same thickness and colors different from each other, since the support structures are of substantially the same thickness, the number of the support structures and the injection rate of medicine can be easily matched to each other, and since they have different colors, it is possible to easily grasp the number of support structures.

In order to achieve the above object, a method for adjusting an injection rate of medicine according to the present invention is featured in that the method for adjusting an injection rate of medicine by a medicine quantitative injector including: a main body; an axial member, the section of which is substantially circular, inserted into the corresponding main body and movable in the forward and backward direction by turning; and a gripping member attached to one end of the axial member and having a longer length in the perpendicular direction with respect to the lengthwise direction of the axial member than the outer diameter of the axial member, includes the steps of: fitting the axial member of the medicine quantitative injector in recessed portions of the unit-matching assisting tool for quantitative injection of medicine being provided with a supporting member composed of a plurality of support structures into which the axial member is fitted and in which recessed portions having a smaller gap than the length of the gripping member and the main body in the perpendicular direction with respect to the lengthwise direction of the axial member are formed, respectively, the recessed portions are made continuous and detachably coupled to each other; and adjusting so as to bring the support structures into contact with the main body and the gripping member by turning the axial member.

Even a diabetic patient who suffers from a lowering in eyesight can self-inject a necessary fixed volume of medicine by a unit-matching assisting tool for quantitative injection of medicine according to the present invention.

Even a diabetic patient who suffers from a lowering in eyesight can self-inject a necessary fixed volume of medicine by a method for adjusting an injection rate of medicine according to the present invention.

### BRIEF DESCRIPTION OF THE DRAWINGS

FIG. 1 is a schematic view showing one example of a unit-matching assisting tool for quantitative injection of medicine to which the present invention is applied,

FIG. 2 is a schematic view of a pen-type insulin injector,

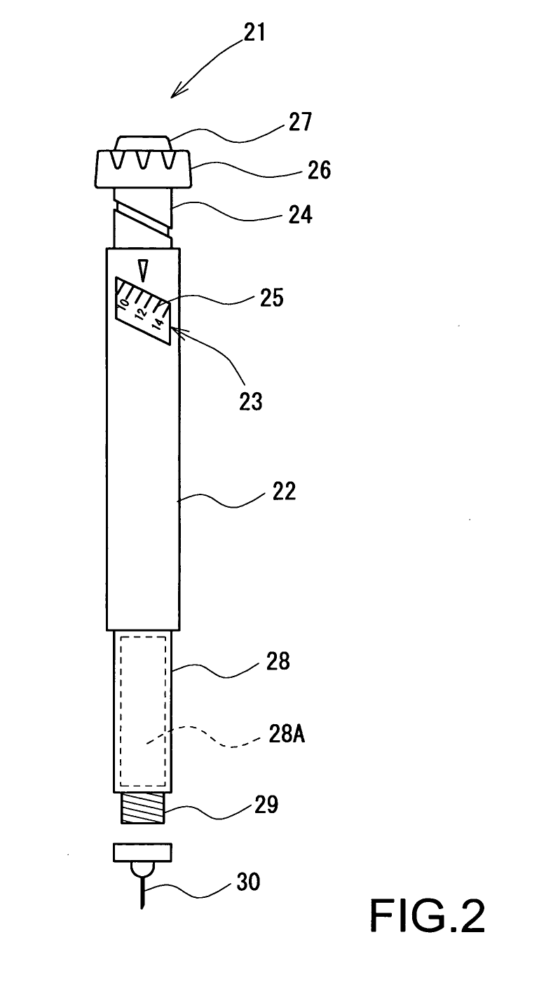

FIG. 3A and FIG. 3B are schematic views describing one example of a method for adjusting an injection rate of medicine by using a unit-matching assisting tool for quantitative injection of medicine according to the present invention, and

FIG. 4 is a schematic view showing a prior art assisting tool for insulin injector.

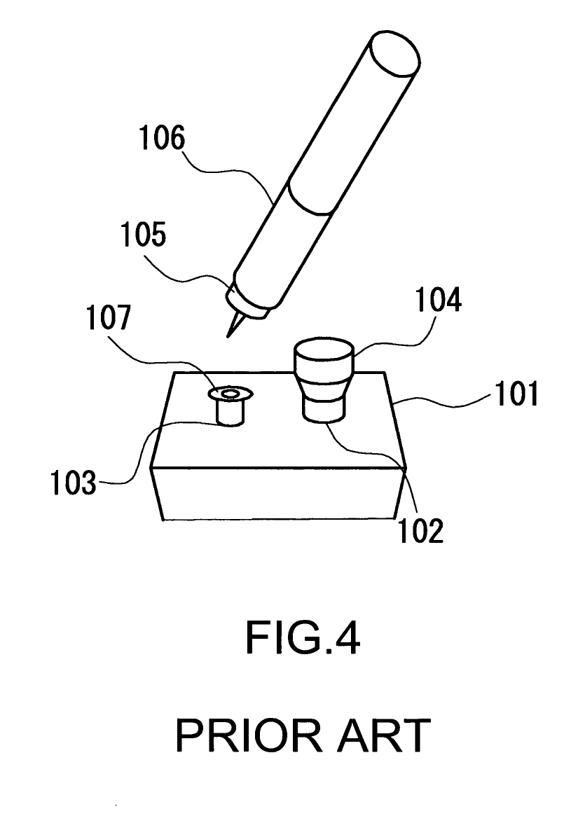

### DETAILED DESCRIPTION OF THE INVENTION

Hereinafter, a description is given of an embodiment of the present invention with reference to the drawings in order to understand the present invention.

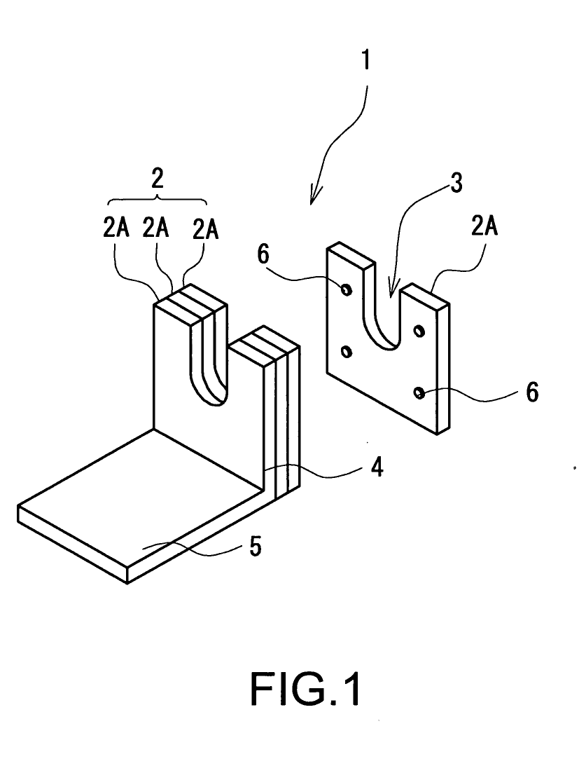

FIG. 1 is a schematic view showing one example of a unit-matching assisting tool for quantitative injection of medicine to which the present invention is applied. In FIG. 1, a unit-matching assisting tool 1 for quantitative injection of medicine according to the present invention is provided with a supporting member 2 composed of a plurality of support structures 2A in which recessed portions 3 having an axial member 24 of a pen-type insulin injector 21 fitted therein, and at the same time, having a smaller gap than the lengths, in the perpendicular direction with respect to the lengthwise direction of the axial member 24, of the gripping member 26 and the main body 22, are respectively formed. The support structures 2A have their recessed portions 3 continued and are detachably coupled to each other by means of magnets 6.

In addition, one of the support structures 2A is provided with the first area 4 in which a recessed portion 3 is formed, and the second area 5 substantially perpendicular with respect to the first area.

Further, the support structures 2A have the same thickness and have colors different from each other.

Herein, if the unit-matching assisting tool for quantitative injection of medicine is provided with a supporting member composed of a plurality of support structures in which recessed portions having an axial member fitted therein, and at the same time having a smaller gap than the respective lengths of the gripping member and the main body in the perpendicular direction with respect to the lengthwise direction of the axial member are respectively formed, and the support structures have their recessed portions continued and are detachably coupled to each other, one of the support structures is not necessarily provided with the first area in which a recessed portion is formed and the second area 5 substantially perpendicular to the first area, and the support structures do not necessarily have the same thickness and have colors different from each other.

In addition, if a unit-matching assisting tool for quantitative injection of medicine according to the present invention is provided with a supporting member composed of a plurality of support structures in which recessed portions having an axial member fitted therein, and at the same time, having a smaller gap than the lengths, in the perpendicular direction with respect to the lengthwise direction of the axial member, of the gripping member and the main body, are respectively formed, and the support structures have their recessed portions continued and are detachably coupled to each other, the support structures are not necessarily detachably coupled to each other by means of magnets, wherein they may be detachably coupled to each other by means of, for example, an adhesive agent and a planar fastener.

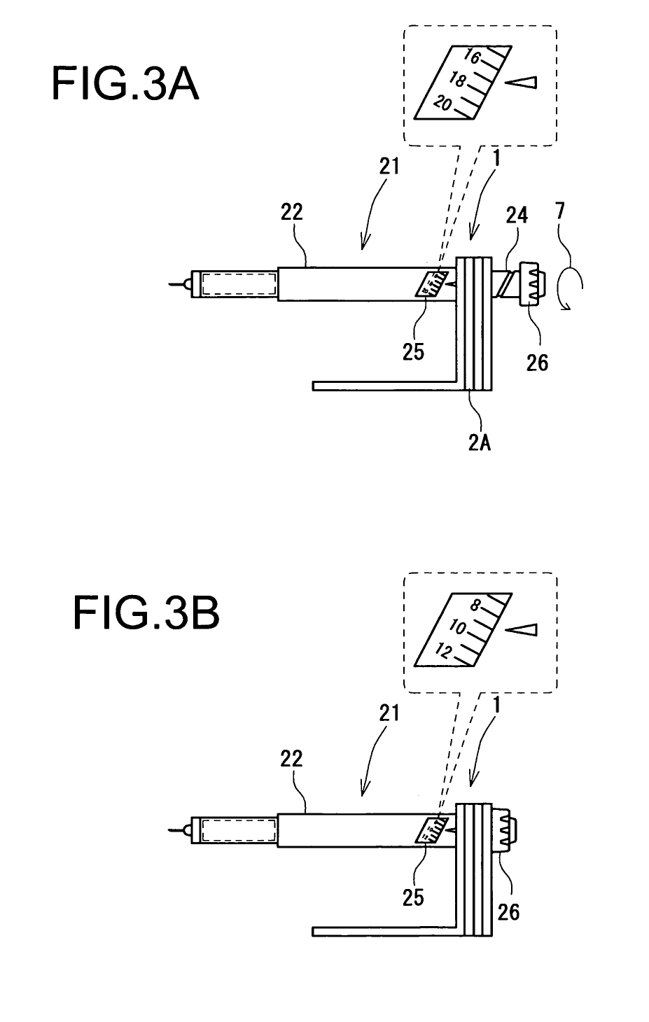

FIG. 3A and FIG. 3B are schematic views describing one example of a method for adjusting an injection rate of medicine by using the unit-matching assisting tool for quantitative injection of medicine according to the present invention.

First, by turning right the gripping member 26 of the insulin injector 21, the axial member 24 is moved in the direction opposite to the main body 22 (that is, the backward direction).

Next, the axial member 24 is fitted in the recessed portions formed in the support structures 2A of the unit-matching assisting tool 1 for quantitative injection of medicine, and the gripping member 26 is turned left in the turning direction 7 to move the axial member 24 in the (forward direction) to the main body 22 (Refer to FIG. 3A).

And, the gripping member 26 is turned left in the turning direction 7 until the support structures 2A are brought into contact with the main body 22 and the gripping member 26, thereby moving the axial member 24 in the forward direction (Refer to FIG. 3B).

In this state, indication of the dial 25 means the injection rate of medicine. That is, in a state of the insulin injector 21 shown in FIG. 3A, the indication of the dial 25 is 17 units, and in the state of the insulin injector 21 shown in FIG. 3B, the indication of the dial 25 is 9 units. That is, the injection rate can be adjusted only by bringing the main body 22 and the gripping member 26 into contact with the support structures 2A.

Thus, since the unit-matching assisting tool for quantitative injection of medicine according to the present invention is provided with a supporting member composed of a plurality of support structures in which recessed portions having an axial member fitted therein, and at the same time, having a smaller gap than the lengths, in the perpendicular direction with respect to the lengthwise direction of the axial member, of the gripping member and the main body, are respectively formed, the supporting member can be brought into contact with the gripping member and the main body. Also, since the support structures have their recessed portions continued and are detachably coupled to each other, the number of the support structures can be increased or decreased. Therefore, since the gripping member and main body of the medicine quantitative injector are brought into contact with the supporting member, and the position where the axial member is no longer moves toward the main body can be determined to be the injection rate, even a diabetic patient who suffers from a lowering in eyesight can self-inject a necessary fixed volume of medicine.

In addition, since at least one of the support structures is provided with the first area having the recessed portion formed therein and the second area substantially perpendicular with respect to the first area, the supporting member is placed on a desk or a table, the axial member of the medicine quantitative injector can be fitted in the recessed portions, it is possible even for a patient who is weak in hand force to easily adjust the injection rate.

Further, since the support structures have substantially the same thickness and colors different from each other, the number of support structures and the injection rate of medicine can be easily matched to each other by virtue of the same thickness, and it is possible to easily grasp the number of the support structures by virtue of different colors.

1. A unit-matching assisting tool for quantitative injection of medicine having a medicine quantitative injector fitted therein and assisting in the quantitative injection of medicine, the medicine quantitative injector including: a main body; an axial member, the section of which is substantially circular, inserted into the corresponding main body and movable in the forward and backward direction by turning; and a gripping member attached to one end of the axial member and having a longer length in the perpendicular direction with respect to the lengthwise direction of the axial member than the outer diameter of the axial member, and
the unit-matching assisting tool further including a supporting member composed of a plurality of support structures into which the axial member is fitted and in which have recessed portions having a smaller gap than the lengths of the gripping member and the main body in the perpendicular direction with respect to the lengthwise direction of the axial member,
wherein the support structures are detachably coupled with each other, in which the recessed portions are made continuous.

2. The unit-matching assisting tool for quantitative injection of medicine according to claim 1,
wherein at least one of the support structures is provided with a first area having the recessed portion formed therein and a second area substantially perpendicular with respect to the first area.

3. The unit-matching assisting tool for quantitative injection of medicine according to claim 1, wherein the support structures have substantially the same thickness and colors different from each other.

4. A method for adjusting an injection rate of medicine by a medicine quantitative injector including: a main body; an axial member, the section of which is substantially circular, inserted into the corresponding main body and movable in the forward and backward direction by turning; and a gripping member attached to one end of the axial member and having a longer length in the perpendicular direction with respect to the lengthwise direction of the axial member than the outer diameter of the axial member, comprising the steps of:
fitting the axial member of the medicine quantitative injector in recessed portions of the unit-matching assisting tool for quantitative injection of medicine being provided with a supporting member composed of a plurality of support structures into which the axial member is fitted and in which recessed portions having a smaller gap than the length of the gripping member and the main body in the perpendicular direction with respect to the lengthwise direction of the axial member are formed, respectively, the recessed portions are made continuous and detachably coupled to each other; and
adjusting so as to bring the support structures into contact with the main body and the gripping member by turning the axial member.

5. The method for adjusting an injection rate of medicine according to claim 4,
wherein at least one of the support structures is provided with a first area having the recessed portion formed therein and a second area substantially perpendicular with respect to the first area, and the second area is seated.
